(19) United States
(12) Patent Application Publication
(10) Pub. No.: US 2015/0335596 A1
(43) Pub. Date: Nov. 26, 2015

(54) 1-(2-FLUOROBIPHENYL-4-YL)-CYCLOPROPANECARBOXYLIC ACID DERIVATIVES FOR THE TREATMENT OF DOWN'S SYNDROME

(71) Applicant: CHIESI FARMACEUTICI S.P.A., Parma (IT)

(72) Inventors: Bruno Pietro IMBIMBO, Parma (IT); Laura CALZÀ, Parma (IT); Luciana Giardino, Parma (IT)

(73) Assignee: CHIESI FARMACEUTICI S.P.A., Parma (IT)

(21) Appl. No.: 14/719,784

(22) Filed: May 22, 2015

(30) Foreign Application Priority Data
May 26, 2014 (EP) 14169849.8

(51) Int. Cl. A61K 31/192

(52) U.S. Cl. CPC A61K 31/192

(57) ABSTRACT

Derivatives of 1-(2-fluorobiphenyl-4-yl)-cyclopropanecarboxylic acid are useful for improving the cognitive capacity of patients with intellectual disabilities, an IQ of less than 85, diagnosed with mental retardation, and, most specifically, those with Down's syndrome.

### CROSS REFERENCES TO RELATED APPLICATIONS

This application claims priority to European Patent Application No. 14169849.8 filed on May 26, 2014, which is incorporated herein by reference in its entirety.

### BACKGROUND OF THE INVENTION

1. Field of the Invention

The present invention relates to 1-(2-fluorobiphenyl-4-yl)-cyclopropanecarboxylic acid derivatives for use in improving the cognitive capacity of patients with intellectual disabilities, an IQ of less than 85, diagnosed with mental retardation, and, most specifically, those with Down's syndrome. In particular, the present invention relates to the use of the afore-mentioned derivatives for the prevention and/or treatment of cognitive symptoms in patients affected by Down's syndrome.

2. Discussion of the Background

Down's syndrome (hereinafter DS) is the most common genetic source of mental retardation. It is caused by the presence of an extra copy of human chromosome 21 (i.e. trisomy 21, HSA 21), which includes the genes SOD-1, BACE-2, APP, and S100b. The neuropathology of DS is complex and includes decreased brain weight, decreased neuronal number, abnormal neuronal differentiation, and structural changes in dendritic spines.

Nervous system involvement, which affects patients throughout the lifespan, results in deficits involving learning, memory, language, and movement. Besides neurodevelopmental alterations, DS is characterized by increased mortality rates, both during early and later stages of life, and age-specific mortality risk remains higher in adults with DS compared with the overall population of people with mental retardation and with normal populations. Causes of higher mortality rates later in life may be due to a number of factors, two of which are an increased risk for Alzheimer's disease (hereinafter AD) and an apparent tendency toward premature aging.

In fact, a distinct feature of DS is the onset of Alzheimer's disease (AD)-like pathology by middle age. Virtually all individuals with DS develop sufficient neuropathology for a diagnosis of AD by the age of 40 years. It has not yet been fully characterized the gene(s) responsible for the DS neuropathology, however the development of AD in DS may be related to the overexpression of the amyloid beta precursor protein (AbetaPP), being APP gene on the trisomic 21 chromosome. In DS patients plasma levels of Abeta40 and Abeta42 are increased (Schupf N et al., Neurosci Lett 2001, 301, 199-203, which is incorporated herein by reference in its entirety). Furthermore, diffuse deposits of the toxic and less soluble Abeta42 form were observed in some DS brains during childhood (Lott I T et al., Neurobiol Aging 2005, 26, 383-389, which is incorporated herein by reference in its entirety) and, in contrast with AD, amyloid deposition was widespread in all of the cortical areas investigated and was observed much earlier than neurofibrillary tangle formation (Hof P R et al., Arch Neurol 1995, 52, 379-391, which is incorporated herein by reference in its entirety). The time course of neurofibrillary tangle formation in DS displays regional patterns comparable with those observed in aging and AD with layer II of the entorhinal cortex being affected first in DS, followed by the hippocampus proper and neocortex. The oldest patients with DS had neurofibrillary tangle densities sometimes higher than in patients with AD.

Moreover, in adults with DS, several markers of inflammation are present at higher levels than in younger individuals, particularly in association with Abeta deposits, and this may be due in part to gene over expression. Inflammation may be a major contributor to the acceleration phase of AD pathogenesis, in DS typically observed between the ages of 40 and 50 years, but may also make a life-long contribution leading to pathological aging. Several genes encoding for inflammatory factors are present on chromosome 21 and are overexpressed and, as in AD patients, cytokines are significantly increased in DS compared to age-matching subjects (Lott et al., Neurobiol Aging 2005; 26: 383-389, which is incorporated herein by reference in its entirety).

From a molecular point of view, a marked alteration in APP processing and Aβ trafficking has been demonstrated in cortical DS astrocytes and neurons (Busciglio J et al., Neuron 2002; 33: 677-688, which is incorporated herein by reference in its entirety) with increased levels of APP and C99, reduced levels of secreted APP (sAPP) and C83, and intracellular accumulation of insoluble Aβ42.

Similar CNS abnormalities have been described in mouse models of DS. In particular, the Ts65Dn mouse, the most widely used model of DS, possess three copies of the segment of mouse chromosome 16 (MMU16) orthologous (80% genes) to the critical region of human chromosome 21 (HSA21) thought to be responsible for the phenotype of DS. The third copy of the distal region of MMU 16 spans from the APP to Mx1 genes, resulting in increased APP gene dosage. These mice have abnormal forebrain and cerebellar development, defects in synapse formation and neurophysiology, and behavioural deficits. These mice recapitulate many characteristics of DS and AD, including age-specific cognitive decline, neuronal cell loss, and decreased levels of nerve growth factor (NGF). This partially trisomic mouse also have elevated expression of APP and AP forms, but plaque formation and tau accumulation have not always been observed (Kern D S et al., Cell Transplant 2011; 20: 371-379, which is incorporated herein by reference in its entirety).

Starting at 10 months of age, brain APP levels were increased proportional to the APP gene dosage imbalance reflecting increased APP message levels in Ts65Dn mice, while at 4 months no difference were observed (Choi J H K et al., Nature 2009; 409: 860-921, which is incorporated herein by reference in its entirety).

From the point of view of cognitive performance, Down's subjects suffer from mental retardation and accelerated aging prone to Alzheimer-type dementia (Schupf N et al., Neurology 2010; 75: 1639-1644, which is incorporated herein by reference in its entirety). Mental retardation in DS appears to be related to severe neurogenesis abnormalities during critical phases of brain development. Recent lines of evidence of the mouse model for DS have shown a defective responsiveness to Sonic Hedgehog (Shh), a potent mitogen that controls cell division during brain development, suggesting involvement of the Shh pathway in the neurogenesis defects of DS (Trazzi S et al., Human Mol Genet 2011; 20: 1560-1573, which is incorporated herein by reference in its entirety). It is therefore possible that abnormalities in neuronal generation, migration, allocation and terminal differentiation may underlie the cognitive phenotype of DS.

Chakrabarti L et al (Nature Neurosci 2010; 13: 927-936, which is incorporated herein by reference in its entirety) demonstrated a complex set of neurogenesis defects in multiple brain regions leading to an excitation/inhibition imbalance in the Ts65Dn forebrain. Furthermore, their results implicate two genes that are triplicated in Down's syndrome and in Ts65Dn mice, Olig1 and Olig2, in the defective ventral neurogenesis. These results also reveal a broader determinative role of Olig1 and Olig2 genes on ventral telencephalic neurogenesis. Thus, although it is well established that Olig1 and Olig2 regulate multiple steps of neuron and glia formation in the CNS37, these transcription factors also affect the rate of neuron production from the medial ganglionic neuron of eminences (MGE) of the ventral telencephalon, under normal conditions and are therefore necessary factors for generating the proper ratio of excitatory and inhibitory neurons in the neocortex. Moreover, the genesis of the pool of undifferentiated precursor cells in the dentate gyrus of the hippocampus is impaired, so that neurogenesis in adulthood is also compromised (Bartesaghi R et al., Rev Neurosci 2011; 22: 419-455, which is incorporated herein by reference in its entirety).

Since DS subjects develop AD pathology at a younger age compared to AD subjects, this is also part of the cognitive impairment and it worsens in middle and old age. The notion that almost 100% of DS subjects are destined to AD pathology, offers the unique opportunity of pre-treatment in order to prevent/delay the onset of AD like pathology.

In summary, notwithstanding that some correlation has been reported in the prior art between DS and AD, there are fundamental genetic differences between an individual with trisomy 21 to non-trisomic people as well as fundamental anatomical and cognitive differences between people with those diseases.

Most evident is that DS is due to a specific genetic anomaly, comprising a third copy of the genetic contents of chromosome 21 (compared to two in the non-DS population) whereas AD is a neurodegenerative disease of largely unknown cause except for the less than 5% of cases caused by variations in one of about 6 genes. In addition, the overall brain morphology of the person with DS is different in many aspects, two of which are the smaller size of many parts of the DS brain and fewer neuronal cells in general. Using positron emission tomography (PET), researchers have shown that the neurophysiology of an aging DS brain differs from that of an AD brain that does not have DS. Specifically, the PET imaging showed higher levels of probe binding in at least two regions of the DS brain relative to the AD brain (Nelson et al., Prog Brain Res 2012; 197: 101-121, which is incorporated herein by reference in its entirety). Furthermore, DS is a condition present at birth whereas AD is a disease of aging. Finally, DS cognitive defects do not progress in contrast with those associated with AD which typically progress throughout the 6-15 years prior to demise.

In view of the above considerations, because neither the DS genotype nor phenotype is the same as that of AD, it cannot be predicted that their respective cognitive deficits could be rescued by the same class of compounds.

The compound 1-(3′,4′-dichloro-2-fluorobiphenyl-4-yl)cyclopropane carboxylic acid, also quoted as CHF 5074, was first disclosed in WO 2004/074232, which is incorporated herein by reference in its entirety, as a therapeutic agent for the treatment of neurodegenerative diseases. It is currently under development for the treatment of the early stages of AD.

### SUMMARY OF THE INVENTION

Accordingly, it is one object of the present invention to provide novel compounds, compositions, and therapeutic methods for improveing the cognitive capacity of patients with intellectual disabilities, an IQ of less than 85, diagnosed with mental retardation, and, most specifically, those with Down's syndrome (DS).

These and other objects, which will become apparent during the following detailed description, have been achieved by the inventors' discovery that compounds of general formula (I):

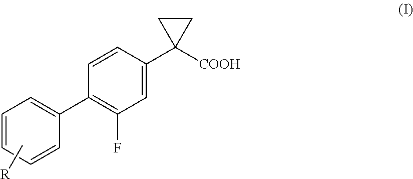

### Wherein:

R represents one or more groups, which can be the same or different from each other, independently selected from halogen atoms, preferably chlorine;

are useful for the prevention and/or treatment of cognitive symptoms in patients affected by Down's syndrome.

Preferably, the compound of formula (I) is 1-(3′,4′-dichloro-2-fluorobiphenyl-4-yl)cyclopropanecarboxylic acid also known with the code CHF 5074.

The present invention also provides the use of the compounds of general formula (I) in the manufacture of a medicament for the prevention and/or treatment of cognitive symptoms in patients affected by Down's syndrome.

In a further aspect, the present invention provides a therapeutic method for improving the cognitive capacity of patients with intellectual disabilities, an IQ of less than 85, diagnosed with mental retardation, and, most specifically, those with Down's syndrome (DS), said method comprising administering an effective amount of a compound of general formula (I), including polymorphs, pharmaceutically acceptable salts and prodrugs thereof.

It has now been found that CHF 5074 and related compounds can effectively be used for the prevention and/or treatment of cognitive symptoms in patients affected by Down's syndrome.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the design of the study in Ts65Dn mice.

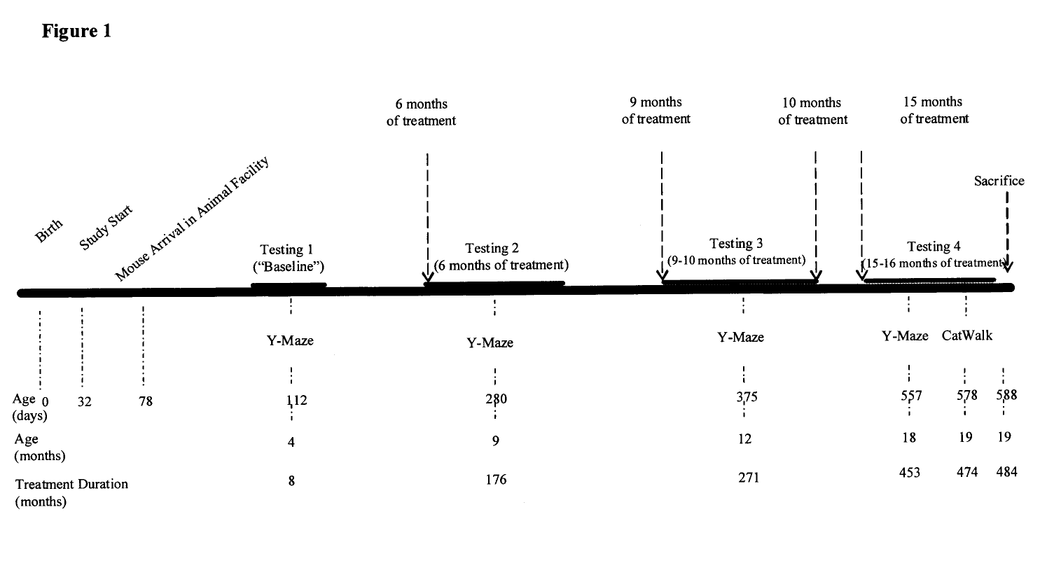

FIG. 2 shows the beneficial effects of long-term treatment with CHF 5074 on some of the age-dependent behavioural and motor abnormalities of Ts65Dn mice.

FIGS. 3 A and B shows the beneficial effects of long-term treatment with CHF 5074 on some of the age-dependent behavioural and motor abnormalities of Ts65Dn mice.

FIGS. 4 A and B illustrates the effects long-term treatment with CHF 5074 on glial cell population (microglia and astrocytes).

FIGS. 5 A and B illustrates the effects of long-term treatment with CHF 5074 on APP metabolism.

FIGS. 6 A and B illustrates the effects of long-term treatment with CHF 5074 on APP metabolism.

FIGS. 7 A, B, C, and D illustrates the effects of long-term treatment with CHF 5074 on APP metabolism.

FIG. 8 illustrates the effects of long-term treatment with CHF 5074 on APP metabolism.

FIG. 9 shows the effects of long-term treatment with CHF 5074 on amyloid precursor protein intracellular domain (AICD) levels in hippocampus.

FIGS. 10 A, B, and C illustrates the effects long-term treatment with CHF 5074 on hippocampal synaptology.

### DETAILED DESCRIPTION OF THE PREFERRED EMBODIMENTS

It can be appreciated that, within the compounds of general formula (I), the phenyl ring bears one or more halogen atoms therein referred to as R groups. From the above, it is clear to the skilled person in the art that any of the said halogen atoms, the same or different from each other, may be thus present in any possible free position of the phenyl ring itself.

The term “halogen atoms” includes fluorine, chlorine, bromine, and iodine.

The term “polymorphs” refers to a different crystal structure of the same solid substance. They exhibit different melting point, solubility (which affect the dissolution rate of the drug and consequently its bioavailability in the body), X-ray crystal and diffraction pattern.

The term “prodrug” refers to a substance administered in an inactive form that is then metabolized in the body in vivo into the active compound with the aim of optimizing absorption, distribution, metabolism, and excretion. In particular, in the context of the present application, prodrugs are used to improve the CNS drug level, with poor crossing of the blood brain barrier usually being the limiting factor.

The term “prevention” refers to the use for progression-slowing and/or onset delaying the cognitive defects.

When the tem “prevention” is used in connection to the “Down's Syndrome”, it should be read as referred to the use for progression-slowing and/or onset delaying the cognitive defects associated to said syndrome.

The term “treatment” refers to the use for curing, symptom-allievating, symptom-reducing the cognitive defects.

The term “IQ” refers to the intelligence quotient. It is a score derived from one of several standardized tests designed to assess intelligence. When current IQ tests are developed, the median raw score of the norming sample is defined as IQ 100 and scores each standard deviation (SD) up or down are defined as 15 IQ points greater or less, although this was not always so historically. By this definition, approximately 95 percent of the population scores an IQ between 70 and 130, which is within two standard deviations of the mean.

There are a variety of individually administered IQ tests in use in the English-speaking world. The most commonly used individual IQ test series is the Wechsler Adult Intelligence Scale for adults and the Wechsler Intelligence Scale for Children for school-age test-takers. Other commonly used individual IQ tests include the current versions of the Stanford-Binet, Woodcock-Johnson Tests of Cognitive Abilities, the Kaufman Assessment Battery for Children, the Cognitive Assessment System, and the Differential Ability Scales. The standard deviation (SD) of IQ is approximately 15 points and mean±1 SD (100±15) includes about ⅔ of the population's performance. IQ scores can differ to some degree for the same person on different IQ tests, so a person does not always belong to the same IQ score range each time the person is tested.

Thus, in one embodiment, the subject has an IQ of less than 85 as measured by any one of the following IQ tests: the Wechsler Adult Intelligence Scale for adults, the Wechsler Intelligence Scale for Children for school-age test-taker, the Stanford-Binet, Woodcock-Johnson Tests of Cognitive Abilities, the Kaufman Assessment Battery for Children, the Cognitive Assessment System, and the Differential Ability Scales.

The present invention provides compounds of formula (I):

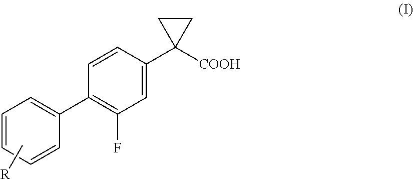

wherein R has the above reported meaning for use in improving the cognitive capacity of patients with intellectual disabilities, an IQ of less than 85, diagnosed with mental retardation, and, most specifically, those with Down's syndrome (DS).

Advantageously, R represents a chlorine atom, and preferably the compound of formula (I) is 1-(3′,4′-dichloro-2-fluorobiphenyl-4-yl)cyclopropanecarboxylic acid, hereinafter quoted with the code CHF 5074.

The compounds of general formula (I) may be prepared according to the procedures described in WO 2009/149797, which is incorporated herein by reference in its entirety.

Said compounds may advantageously be used in any form, amorphous or crystalline and solvates or hydrates thereof. Preferably, they are used in crystalline form.

In view of the close relationship between the compounds of general formula (I) in free acid form and those on the form of salts, the invention is also directed to the use of pharmaceutically acceptable salts thereof.

Pharmaceutically acceptable salts according to the invention include those formed with both common organic and inorganic bases.

The compounds of formula (I) may also be administered in form of a prodrug.

Suitable prodrugs may be esters with common alcohols such as ethanol or polyalcohols such as sorbitol, with sugars such as glucose, or with sugar acids such as ascorbic acid.

In particular, since in Down's syndrome, CNS is the most severe affected tissue, prodrugs which are able of crossing the blood brain barrier such as those disclosed in WO 2006/016219, which is incorporated herein by reference in its entirety, may be advantageously utilized.

The compound of formula (I) may be used alone or in combination with other active ingredients such as N-methyl-D-aspartate (NMDA) receptors receptor antagonists, preferably with memantine.

The compounds of formula (I), may be combined with one or more pharmaceutically acceptable carriers or excipients to provide suitable pharmaceutical compositions.

The pharmaceutically acceptable carriers or excipients may be advantageously selected from the group consisting of diluents, wetting agents, emulsifying agents, binders, coatings, fillers, glidants, lubricants, disintegrants, preservatives, stabilizers, surfactants, pH buffering substances, flavoring agents and similar ones. Comprehensive guidance on pharmaceutical excipients is given in Remington's Pharmaceutical Sciences Handbook, XVII Ed. Mack Pub., N.Y., U.S.A., which is incorporated herein by reference in its entirety.

The pharmaceutical compositions of the present invention may be formulated for administration by any convenient route, e.g. by oral, parenteral, topical, inhalation, buccal, nasal, rectal, vaginal, transdermal administration. Suitable dosage forms can include tablets, capsules, caplets, lozenges, suppositories, solutions, emulsions, suspensions, syrups, ointments, creams, oils, and powders. Preferably, the pharmaceutical compositions of the invention will be administered orally using appropriate dosage forms, such as capsules, tablets, caplets, etc.

The dosage of the compounds of formula (I) and of their salts and prodrugs can vary within wide limits depending on the nature of the disease to be treated, the type of patient, and the mode of administration. A person skilled in the art can determine a therapeutically effective amount for each patient and thereby define the appropriate dosage. When the preferred compound of the invention is administered by oral route to humans, a typical daily dosage might fall within the range of 10 mg to 2000 mg advantageously from 50 to 1000 mg, preferably from 100 to 500, mg administered in a single or multiple daily dosage units. Thus, a single dose of the pharmaceutical preparations of the invention conveniently comprises from 50 to 1000 mg of CHF 5074 or salt or prodrug thereof.

In a preferred embodiment, the compounds of the present invention may be of use for improving the cognitive capacity of patients affected by Down's syndrome, preferably young patients having an age of 2 to 10 years.

They may be also of use for preventing and/or delaying the onset or slowing the cognitive defects in said disease.

Other features of the invention will become apparent in the course of the following descriptions of exemplary embodiments which are given for illustration of the invention and are not intended to be limiting thereof.

### EXAMPLES

The objective of this study was to evaluate if chronic treatment with CHF 5074 could revert or ameliorate the behavioural, histopathological and biochemical abnormalities observed in a transgenic mouse model DS.

The study was performed on Ts65Dn male, using the administration of CHF 5074 in the diet (375 ppm, equivalent to approximately 60 mg/kg/day).

The treatment started at 2 to 3 months of age and it was chronically administered in the diet for 15 months, until 19 months of age.

Treatment groups were:
Wild-type mice receiving standard diet (N=20)
Wild-type mice receiving CHF 5074-medicated diet (375 ppm, approximately 60 mg/kg/day) (N=20)
Ts65Dn mice receiving standard diet (N=20)
Ts65Dn mice receiving CHF 5074-mediacted diet (375 ppm, approximately 60 mg/kg/day) (N=20)

The specific objectives of this study were:

1. To evaluate the effects of chronic treatment with CHF 5074 on spatial learning and memory deficit in Ts56Dn mice.

2. To evaluate the effects of chronic treatment with CHF 5074 on locomotion activity abnormalities in Ts56Dn mice.

3. To evaluate the effects of chronic treatment with CHF 5074 on cellular and molecular players for learning and memory in the hippocampus. To verify the possible effect of chronic treatment with CHF 5074 on expression of molecules known to a play critical role in long-term synaptic plasticity and long-term memory in the hippocampus of adult Ts65Dn mice.

4. To evaluate the effects of chronic treatment with CHF5074 on AD-like brain pathology and APP metabolism.

### Methods.

### Animals and Treatments.

Animals were provided by Jackson Laboratory, Bar Harbor, Me., USA) in small cohorts. Ts65Dn segmental male trisomic mice were used. Ts65Dn mice were compared with diploid controls from the same litters.

### Assessments.

Spatial learning and memory were assessed with the Y-maze test according to Stewart S et al., J Alzheimer Dis 2011; 26: 105-126, which is incorporated herein by reference in its entirety.

Locomotion deficits were assessed with the CatWalk technology (gait analysis) according to Faizi M et al., Neurobiol Dis 2011; 43: 397-413, which is incorporated herein by reference in its entirety.

Brain morphology was assessed by immunohistochemistry, immunofluorescence and quantitative morphometry and microdensitometry according to Imbimbo B P et al., J Alzheimer Dis 2010; 20:159-173; Sivilia S et al., BMC Neurosci 2013; 14: 44; and Guidi S et al., Brain 2014; 137: 380-401, all of which are incorporated herein by reference in their entireties. The following proteins were investigated:

Glial fibrillary acidic protein (GFAP) as astrocyte marker in hippocampus;

Ionized calcium binding adaptor molecule 1 (Iba1), as microglia marker in hippocampus;

Synaptophysin. It is a pre-synaptic vesicle membrane protein known to mediate vesicle release in hippocampus;

Vesicular glutamate transporter 1 (VGLUT-1), as marker of glutamatergic synapses in hippocampus;

Vesicular GABA transporter (VGAT), as marker of GABAergic synapses in hippocampus;

Aβ40 and Aβ42 were measured by ELISA in plasma and brain extracts (IBL kit for mouse Aβ40/Aβ42);

Intraneuronal APP was measured by immunohistochemistry and computerized microdensitometry (Chemicon clone 22C11) in cerebral cortex;

Amyloid precursor protein intracellular domain (AICD) (cleavage sites C83 and C99) in hippocampus.

Molecular biology markers were measured in hippocampus and basal ganglia by real-time PCR, according to Massella A et al., BMC Neuroscience 2012; 13: 12; and D'Intino G et al., J Neuroendocrinol 2011; 23: 778-790, both of which are incorporated herein by reference in their entireties.

### Results.

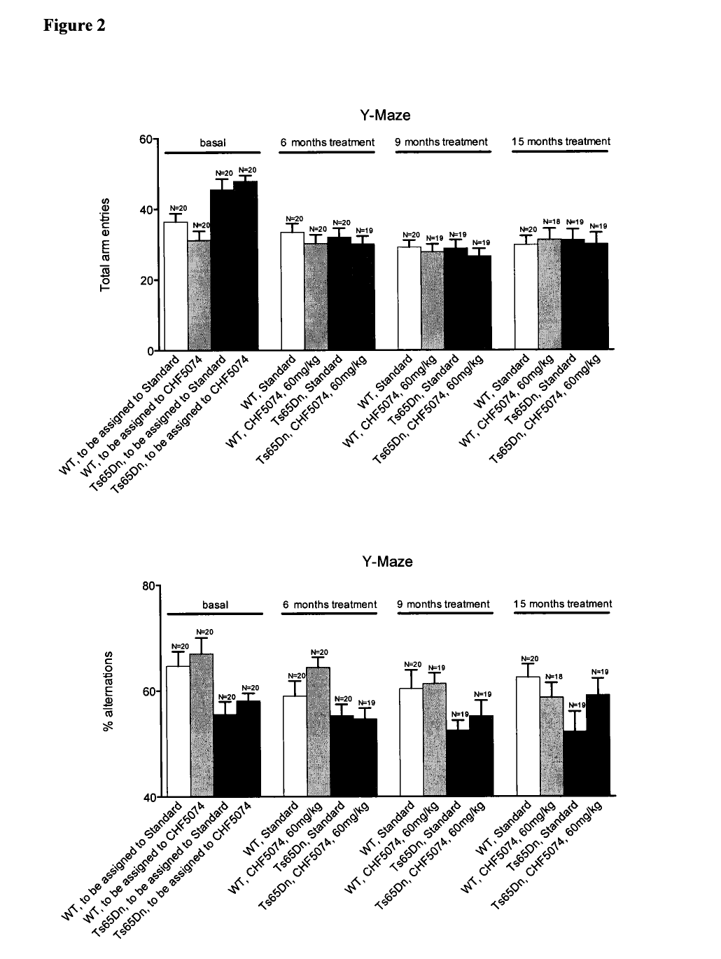

FIG. 2 shows spatial memory, as assessed with the Y maze test before starting CHF 5074 treatment (3.5 months of age) and after 6, 9 and 15 months of treatment. Animals were tested in a single trial Y maze task, to measure arm entries (A) and alternation (B). The percentage of spontaneous alternations is defined as (total alternations/total arm entries-2)×100. Before starting treatment (3.5 months of age), Ts65Dn mice showed a significant deficit in spatial memory compared to wild-type mice as evidenced by low spontaneous alternation (panel B). CHF 5074 treatment progressively normalized spontaneous alternation in Ts65Dn mice and after 15 months of treatment this was not significantly different from that of wild-type mice.

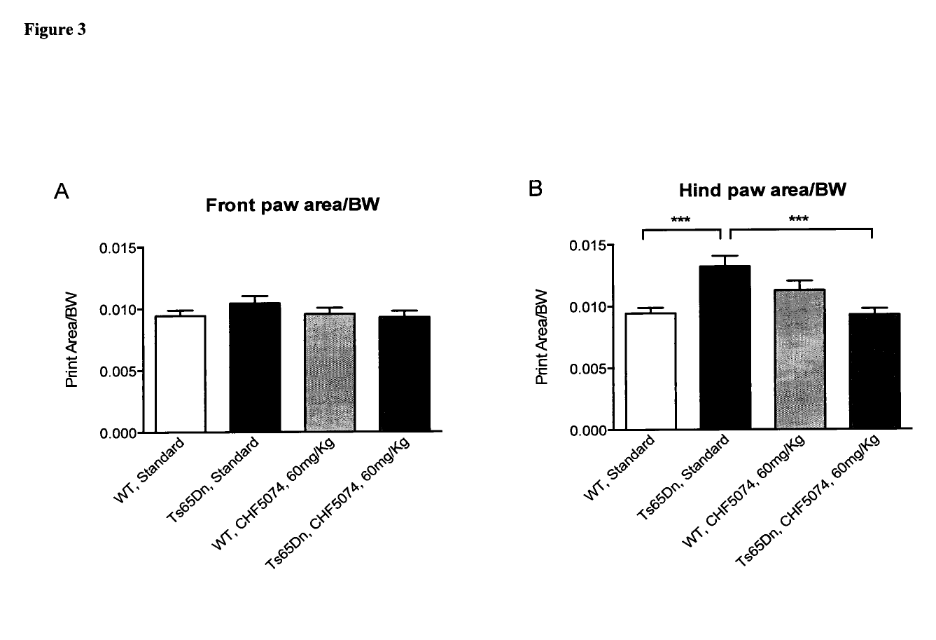

FIGS. 3 A and B shows locomotion performance, as assessed using computerized gait analysis (Cat Walk) in animals after 15 months of treatment (19 months of age). The print area of front and hind paws is presented, as corrected according to the individual animal body weight. While no differences were observed in front paw print area, the hind paw print area of old Ts65Dn mice is increased. CHF 5074 treatment normalized this gait parameter.

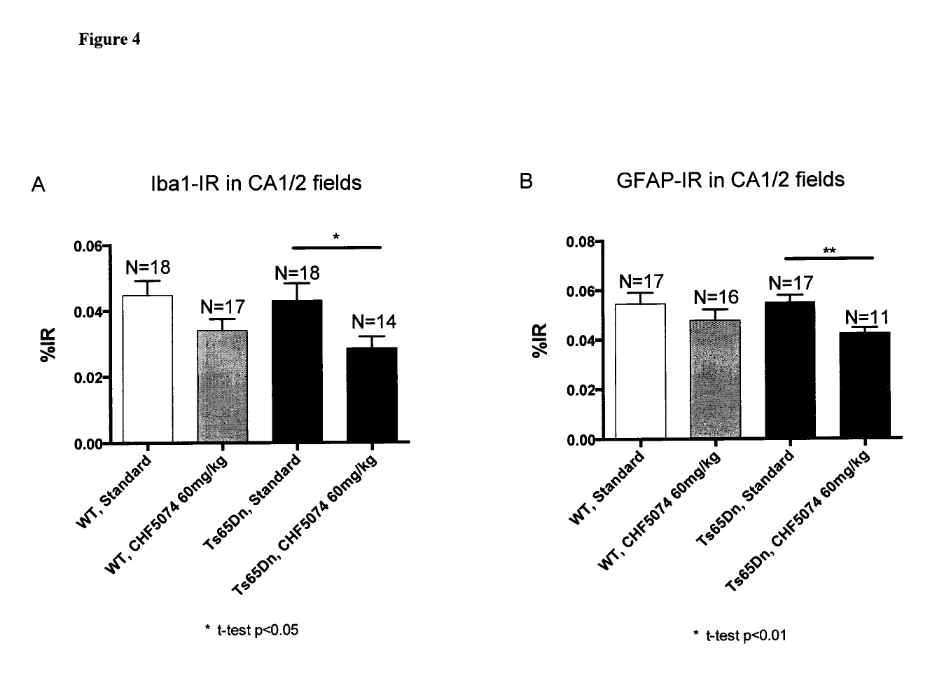

FIGS. 4 A and B shows quantification of activated microglia (Iba1-immunoreactivity) and reactive astrocytes (GFAP-immunoreactivity) in the CA1/2 regions of the hippocampus. Aged Ts65Dn mice treated with CHF 5074 showed a substantial reduction of both Iba1- and GFAP-immunoreactivity compared to Ts65Dn mice receiving standard diet (*p<0.05, **p<0.01). These results indicate that CHF 5074 reduced cellular markers of neuroinflammation in the brain of Ts65Dn mice.

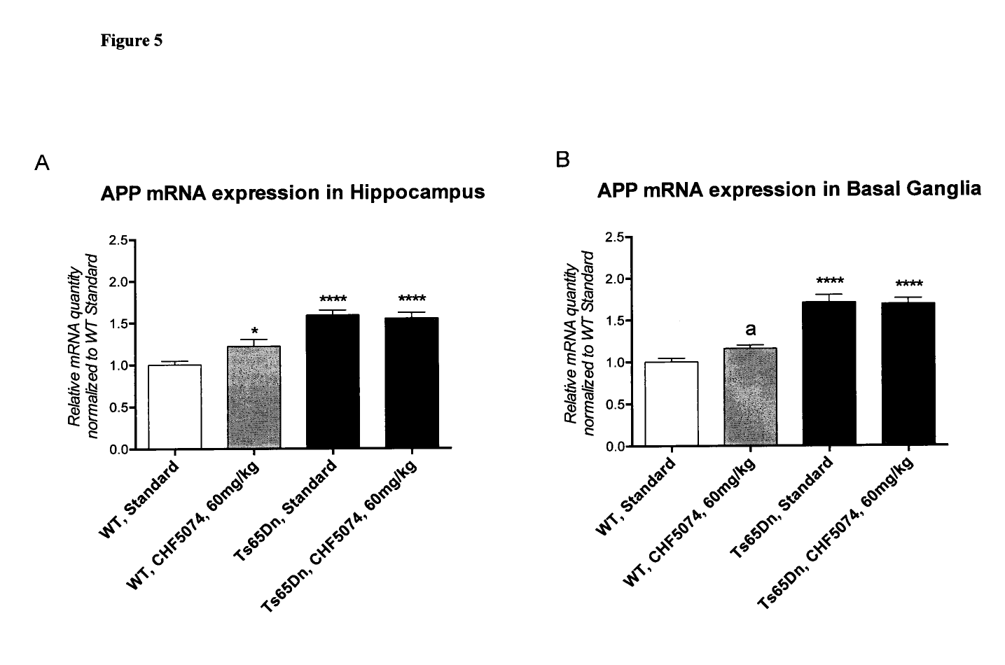

FIGS. 5 A and B illustrates the APP mRNA expression levels in the hippocampus and basal ganglia of wild-type and Ts65Dn mice treated with CHF 5074-medicated diet or normal diet for 15 months (19 months of age). There was a substantial increase in APP mRNA expression levels in Ts65Dn mice compared to wild-type animals. Moreover, CHF 5074 treatment slightly but significantly increased APP mRNA levels in wild-type animals (*p<0.05, ****p<0.0001, ^{a}p<0.1).

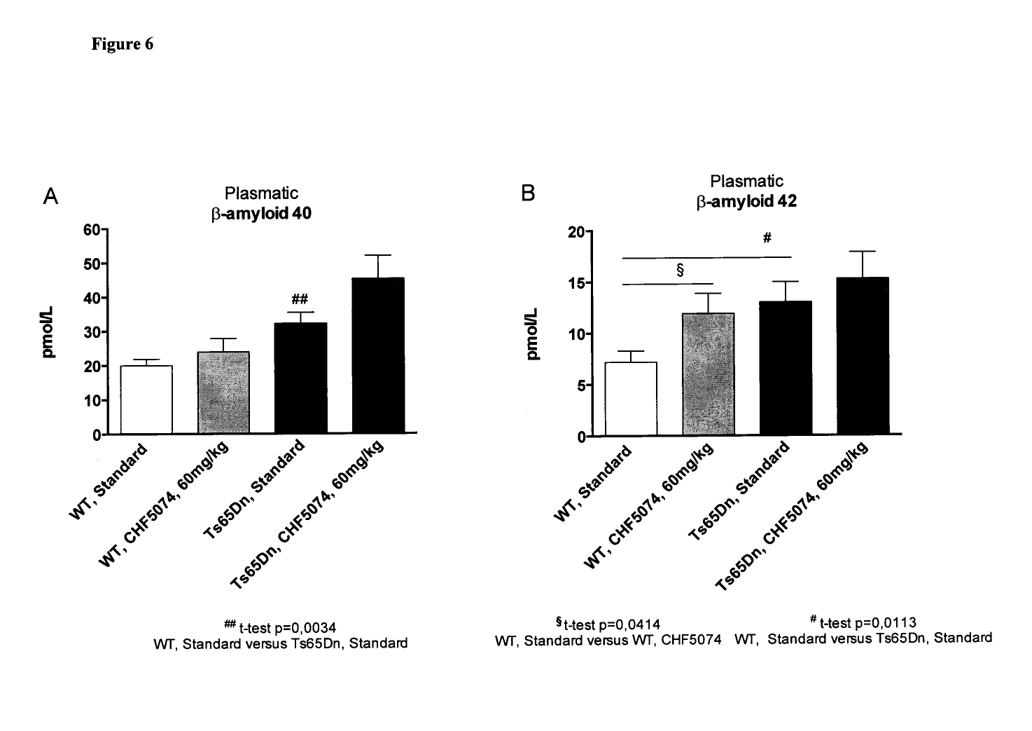

FIGS. 6 A and B illustrates the plasma levels of Aβ40 and Aβ42 peptides in animals after 15 months of treatment (19 months of age). There was a substantial increase in Aβ40 and Aβ42 plasma levels in Ts65Dn mice compared to wild-type animals. CHF 5074 further increased plasma level of both fragment, being this effect significant for Aβ42 in wild-type animals.

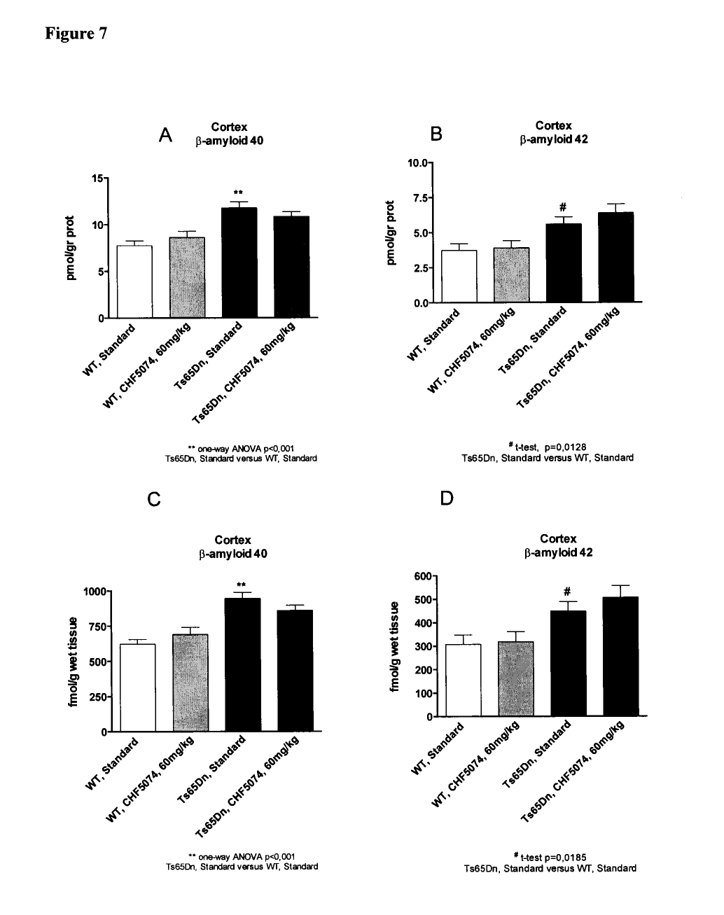

FIGS. 7 A, B, C, and D illustrates the brain (cerebral cortex) levels of Aβ40 and Aβ42 in animals after 15 months of treatment (19 months of age). Results are expressed as pmol/gr proteins (panels A and B), and as fmol/g wet tissue (panels C and D). There was a substantial increase in Aβ40 and Aβ42 brain levels in Ts65Dn mice compared to wild-type animals. CHF 5074 treatment did not modify significantly brain Aβ40 and Aβ42 levels.

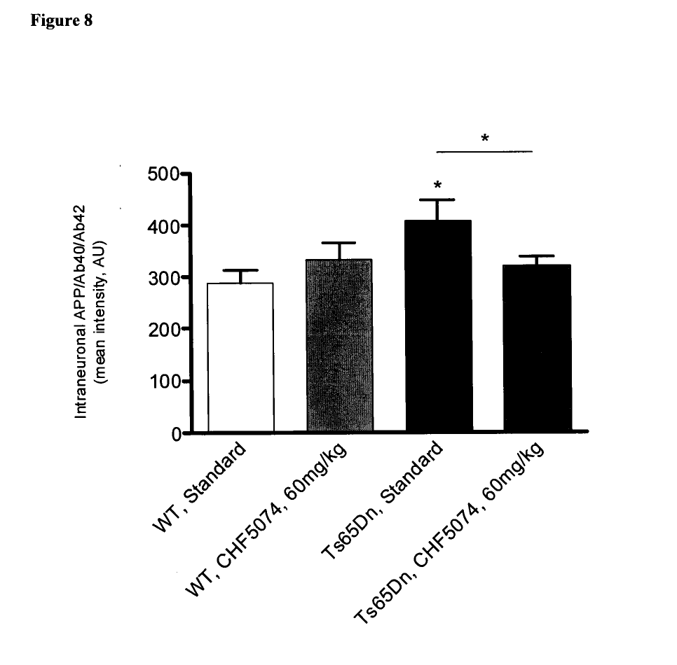

FIG. 8 shows intracellular immunoreactivity of total APP/Aβ40/Aβ42 peptides in cortical neurons. There was a substantial increase in intraneuronal immunoreactivity in Ts65Dn mice compared to wild-type animals and a significant decrease in this immunoreactivity in Ts65Dn mice treated with CHF 5074 compared to control transgenic animals.

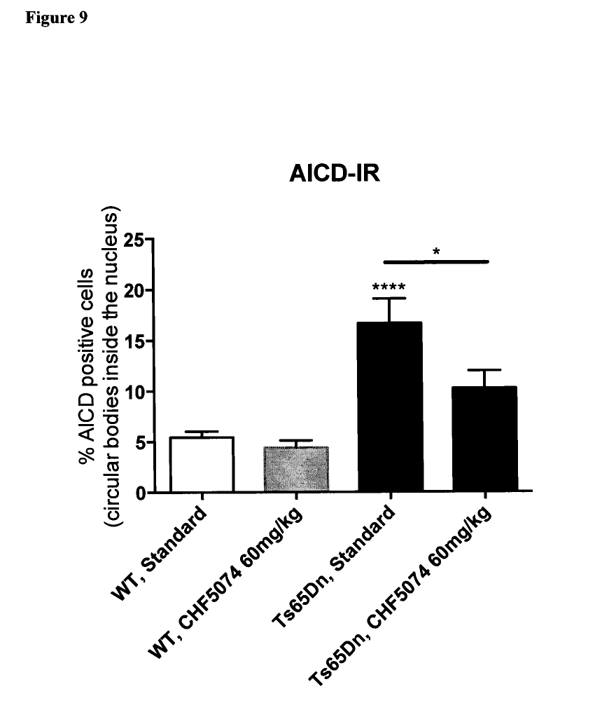

FIG. 9 shows the quantification of AICD (amyloid precursor protein intracellular domain) immunoreactivity (IR) in neurons in the CA1/2 hippocampal regions. Quantification was carried out by counting the percentage of neurons having AICD-positivity. There was a three-fold increase in the percentage of AICD-positive neurons in Ts65Dn mice and this increase was significantly attenuated by CHF 5074 treatment (****p<0.0001).

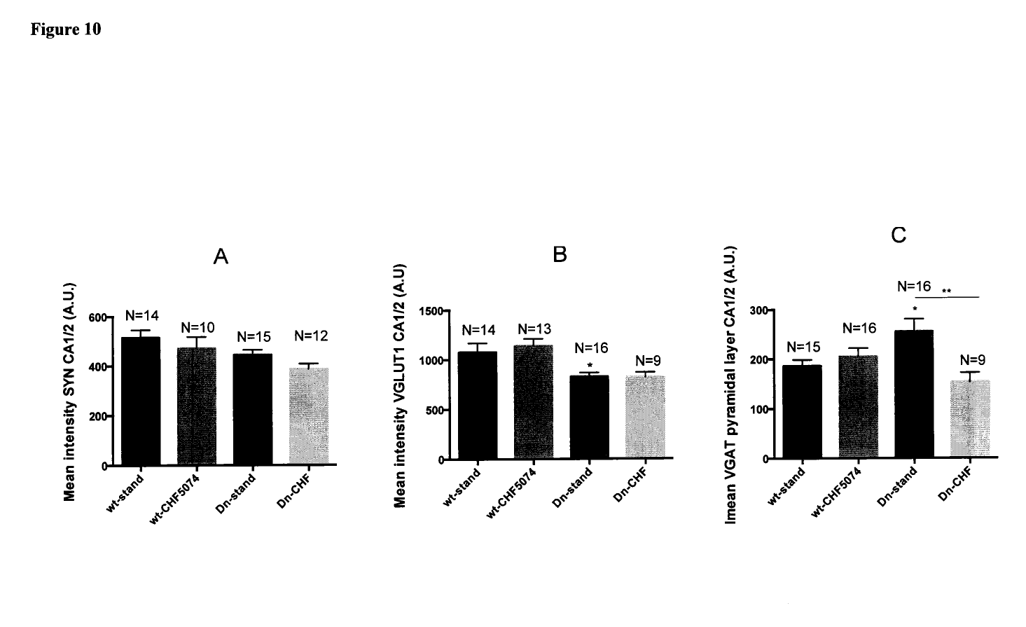

FIG. 10 illustrates main markers of synaptic transmission in the CA1/2 regions of the hippocampus after 15 months of treatment (19 months of age). Panel A reports synaptophys levels and there were no significant differences between experimental groups. Panel B reports the variation of immunostaining for the glutamate transporter VGLUT1 with a substantial decrease in Ts65Dn mice compared to wild-type animals. Panel C reports the variation of immunostaining for the GABA transporter VGAT. There was a substantial increase in Ts65Dn mice compared to wild-type animals and this increase was completely reversed by the CHF 5074 treatment.

The results of this study indicated that prolonged oral treatment with CHF 5074 attenuated spatial memory deficit and completely reversed locomotion deficit in Ts65Dn mice. Histological and biochemical analyses showed that these behavioral improvements were associated to reduced neuroinflammation markers and morphological signs of neuroprotection in hippocampus at the synaptic level (vesicular GABA transporter) in the CHF 5074-treated Ts65Dn mice. Surprisingly, CHF 5074 treatment of Ts65Dn mice significantly attenuated the dramatic increase in amyloid precursor protein intracellular domain (AICD) observed in hippocampal neurons of the control Ts65Dn mice treated with standard diet, suggesting a novel molecular mechanism for the behavioral improvements. Thus, our findings provide evidence for memory and locomotion facilitation of CHF 5074 after chronic treatment in this mouse model. On the basis of these findings, it can be reasonably hypothesized that CHF 5074 and close analogs thereof can be utilized for the treatment of subjects with Down syndrome.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method of improving the cognitive capacity of a patient with an intellectual disability, an IQ of less than 85, and diagnosed with mental retardation, comprising administer to a subject in need thereof a compound of formula (I): 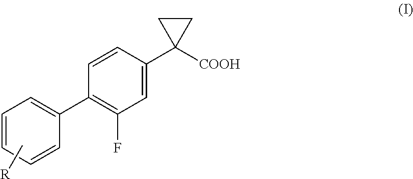
wherein:
R represents one or more groups, which can be the same or different from each other, and are independently a halogen atom.

2. A method according to claim 1, wherein R is one or more chlorine atoms.

3. A method according to claim 1, comprising administering an effective amount of 1-(3′,4′-dichloro-2-fluorobiphenyl-4-yl)cyclopropanecarboxylic acid (CHF 5074) to said subject.

4. A method for the prevention and/or treatment of Down's syndrome, comprising administering to a subject in need thereof an effective amount of a compound of formula (I): 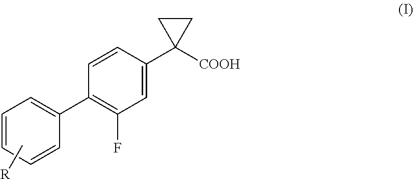
wherein:
R represents one or more groups, which can be the same or different from each other, and are independently a halogen atom.

5. A method according to claim 4, wherein said subject is 2 to 10 years of age.

6. A method according to claim 4, wherein R is one or more chlorine atoms.

7. A method according to claim 5, wherein R is one or more chlorine atoms.

8. A method according to claim 4, comprising administering an effective amount of 1-(3′,4′-dichloro-2-fluorobiphenyl-4-yl)cyclopropanecarboxylic acid (CHF 5074) to said subject.

9. A method according to claim 5, comprising administering an effective amount of 1-(3′,4′-dichloro-2-fluorobiphenyl-4-yl)cyclopropanecarboxylic acid (CHF 5074) to said subject.
